(19) United States
(12) Patent Application Publication
(10) Pub. No.: US 2007/0135754 A1
(43) Pub. Date: Jun. 14, 2007

(54) ELECTRODE ASSEMBLY FOR IONTOPHORESIS FOR ADMINISTERING ACTIVE AGENT ENCLOSED IN NANOPARTICLE AND IONTOPHORESIS DEVICE USING THE SAME

(76) Inventors: Hidero Akiyama, Shibuya-ku (JP); Mizuo Nakayama, Shibuya-ku (JP); Takehiko Matsumura, Shibuya-ku (JP); Akihiko Matsumura, Shibuya-ku (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 11/537,459

(22) Filed: Sep. 29, 2006

Related U.S. Application Data
(60) Provisional application No. 60/730,226, filed on Oct. 24, 2005.

(30) Foreign Application Priority Data
Sep. 30, 2005 (JP) 2005-288148

(51) Int. Cl. A61N 1/30

(52) U.S. Cl. 604/20

(57) ABSTRACT

An electrode assembly for iontophoresis may enable the administration of a non-ionic active agent ionized, or an active agent that is substantially insoluble in water, by iontophoresis. An electrode assembly used for iontophoresis may hold an active agent enclosed in an ionic nanoparticle.

### CROSS-REFERENCE TO RELATED APPLICATION

This application claims the benefit under 35 U.S.C. § 119(e) of U.S. Provisional Patent Application No. 60/730,226, filed Oct. 24, 2005.

### BACKGROUND

1. Field

The present disclosure generally relates to the field of iontophoresis, and to an iontophoresis device that uses the electrode assembly.

2. Description of the Related Art

Iontophoresis refers to a method of delivering an ionic active agent placed on the surface of a biological interface, such as skin or mucosa, of a subject and into the subject's body by use of an electromotive force sufficient to drive the ionic active agent. Refer to JP 63-35266 A for one example of iontophoresis.

Positively charged ions may be driven (transported) into a biological interface on an anode (positive electrode) side of an iontophoresis device, for example. Negatively charged ions may be driven into a biological interface on a cathode (negative electrode) side.

Several iontophoresis devices have been proposed. For example, JP 63-35266 A, JP 04-297277 A, JP 2000-229128 A, JP 2000-229129 A, JP 2000-237327 A, JP 2000-237328 A, WO 03/037425 A1, JP 2004-518707 A, JP 2004-231575 A, and JP 2003-501379 A.

It may be difficult, however, to apply iontophoresis to delivery of an active agent that does not dissociate into ions, that is insoluble in water, and/or that has a high molecular weight. The number of potential active agents deliverable by conventional iontophoresis may thus be limited.

### BRIEF SUMMARY

In one aspect, the present disclosure is directed to an electrode assembly for iontophoresis that may be used to administer active agents that are incapable of dissociating into ions, and/or active agents that are substantially insoluble in water. An active agent may be held enclosed in an ionic nanoparticle.

In one aspect, the present disclosure is directed to an electrode assembly that includes an electrode coupled to a pole of an electric power source device having the same polarity as that of ionic nanoparticles to be delivered, and an electrolyte solution reservoir that holds an electrolyte solution. The electrolyte solution reservoir may be placed adjacent to the electrode, and an ion exchange membrane that selectively passes ions having a polarity opposite that of the ionic nanoparticles may be disposed adjacent to the electrolyte solution reservoir. An active agent solution reservoir that holds the ionic nanoparticles may be placed adjacent to the ion exchange membrane, and an ion exchange membrane that selectively passes ions having the same polarity as that of the ionic nanoparticles may be placed adjacent to the active agent solution reservoir.

In one aspect, the present disclosure is directed to an electrode assembly that includes an active agent solution reservoir in which pores capable of holding and passing the ionic nanoparticle are formed.

In one aspect, the present disclosure is directed to an electrode assembly that includes an ion exchange membrane which selectively passes ions having the same polarity as that of the ionic nanoparticle and which has pores capable of passing the ionic nanoparticle are formed. An ion exchange membrane that selectively passes ions having a polarity opposite that of the ionic nanoparticles may be substantially free of pores capable of passing the ionic nanoparticle.

In one aspect, the present disclosure is directed to an iontophoresis device that includes an electric power source device, and two or more electrode assemblies capable of delivering ionic nanoparticles coupled to the electric power source device. Current control means for controlling current flow to each of the electrode assemblies may also be included.

The active agent administration means may be constructed integrally.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a schematic diagram showing an electrode assembly for iontophoresis according to one illustrated embodiment.

FIG. 2 is a schematic diagram showing an iontophoresis device that includes an electrode assembly for iontophoresis according to one illustrated embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with iontophoresis devices, controllers, voltage or current sources and/or membranes have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “another embodiment” means that a particular referent feature, structure, or characteristic described in connection with the embodiment is included in at least one embodiment. Thus, the appearances of the phrases “in one embodiment,” or “in an embodiment,” or “another embodiment” in various places throughout this specification are not necessarily all referring to the same embodiment. Further more, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to a system for evaluating an iontophoretic active agent delivery including “a controller” includes a single controller, or two or more controllers. It should also be noted that the term “or” is generally employed in its sense including “and/or” unless the content clearly dictates otherwise.

As used herein the term “membrane” means a boundary, a layer, barrier, or material, which may, or may not be permeable. The term “membrane” may further refer to an interface. Unless specified otherwise, membranes may take the form a solid, liquid, or gel, and may or may not have a distinct lattice, non cross-linked structure, or cross-linked structure.

As used herein the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH and ACS also from Tokuyama Co., Ltd.

As used herein, the term bipolar membrane means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules a first rate, and some other molecules a second rate different than the first. In yet further embodiments, the “semi-permeable membrane” may take the form of a selectively permeable membrane allowing only certain selective molecules to pass through it.

As used herein, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled macromolecules (e.g., cylindrical micelles). In some embodiment a gel matrix may include hydrogels, organogels, and the like. Hydrogels refer to three-dimensional network of, for example, cross-linked hydrophilic polymers in the form of a gel and substantially composed of water. Hydrogels may have a net positive or negative charge, or may be neutral.

A used herein, the term “reservoir” means any form of mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution.

A used herein, the term “active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., an active agent, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, an anti-tumor agent. In some embodiments, the term “active agent” further refers to the active agent, as well as its pharmacologically active salts, pharmaceutically acceptable salts, proactive agents, metabolites, analogs, and the like. In some further embodiment, the active agent includes at least one ionic, cationic, ionizable and/or neutral therapeutic active agent and/or pharmaceutical acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. While other active agents may be polarized or polarizable, that is exhibiting a polarity at one portion relative to another portion. For instance, an active agent having an amino group can typically take the form an ammonium salt in solid state and dissociates into a free ammonium ion (NH₄⁺) in an aqueous medium of appropriate pH. The term “active agent” may also refer to neutral agents, molecules, or compounds capable of being delivered via electro-osmotic flow. The neutral agents are typically carried by the flow of, for example, a solvent during electrophoresis. Selection of the suitable active agents is therefore within the knowledge of one skilled in the art.

Non-limiting examples of such active agents include lidocaine, articaine, and others of the -caine class; morphine, hydromorphone, fentanyl, oxycodone, hydrocodone, buprenorphine, methadone, and similar opiod agonists; sumatriptan succinate, zolmitriptan, naratriptan HCI, rizatriptan benzoate, almotriptan malate, frovatriptan succinate and other 5-hydroxytryptamine1 receptor subtype agonists; resiquimod, imiquidmod, and similar TLR 7 and 8 agonists and antagonists; domperidone, granisetron hydrochloride, ondansetron and such anti-emetic active agents; zolpidem tartrate and similar sleep inducing agents; L-dopa and other anti-Parkinson's medications; aripiprazole, olanzapine, quetiapine, risperidone, clozapine and ziprasidone as well as other neuroleptica; diabetes active agents such as exenatide; as well as peptides and proteins for treatment of obesity and other maladies.

As used herein and in the claims, the term “subject” generally refers to any host, animal, vertebrate, or invertebrate, and includes fish, mammals, amphibians, reptiles, birds, and particularly humans.

As used herein, the term “nanoparticle” generally refers to a hollow particle having a diameter of 4 nm to 400 nm and capable of substantially enclosing a substance therein.

The headings provided herein are for convenience only and do not interpret the scope or meaning of the embodiments.

Ionic nanoparticles generally have positively or negatively charged functional groups on a surface film thereof. This charged nature allows ionic nanoparticles to be administered via iontophoresis. For example, a cationic nanoparticle may have a functional group such as —NH3+. The functional group can reinforce an interaction with a negatively charged biological cell. An anionic nanoparticle, by way of comparison, may have a functional group such as a carboxyl group, so interactions with negatively charged biological cell can be suppressed.

Non-limiting examples of materials that may be used to form ionic nanoparticles include PLGA (polylactic acid-glycolic acid) and PLA (polylactic acid). Use of such a material may delay the release of an active agent as the molecular weight of the ionic nanoparticle becomes larger. In addition, the period of time during which an active agent is effective may be lengthened due to sustained release properties found when using such materials. This may help reduce any burden placed on a subject.

Ionic nanoparticles may be coated with lecithin, for example. Coated ionic nanoparticles may accumulate on a portion of a biological interface that is inflamed or the like, thereby enabling targeted delivery. In addition, ionic nanoparticles may be susceptible to deformation, similar to red blood cells, through energization, thus allowing the ionic nanoparticles to pass through and be absorbed by an ion exchange membrane or a biological interface.

Ionic nanoparticles enclosing an active agent therein may be formed by using any of a variety of methods, some of which are outlined below.

The following method may be used when a water soluble active agent is to be enclosed in ionic nanoparticles. The active agent may be dissolved in acetone along with PLGA (or PLA), and zinc acetate may be added to the solution. The resultant may then be added dropwise to water, and lecithin may then be added, thus forming nanospheres.

A specific example follows. One mg of triptorelin may be dissolved into 100 μL of an aqueous solution (pH 7, W1). The solution may then be emulsified with an organic solvent (ethyl acetate or dichloromethane) into which PLGA has been dissolved, and the resultant may be exposed to ultrasonic irradiation. Next, 2 mL of an aqueous solution containing 1% PVA (W2) may be added to the resultant, and the whole may be exposed to ultrasonic irradiation to prepare a W1/0/W2 emulsion. The emulsion can then be diluted with 15 mL of a 0.3% aqueous solution of PVA, and the organic solvent evaporated under reduced pressure. The resultant nanoparticles may be separated by means of ultracentrifugation (13,000×G, 30 minutes). A resulting supernatant may be removed and washed with purified water.

Fine particles prepared by using PLGA50/50 having a large number of free carboxyl groups, each having negative charge. In addition, when prepared fine particles and cetyl butyl ammonium bromide (CTAB) are mixed and stirred overnight, CTAB adsorbs onto the surface of each particle, so the charge of each fine particle becomes positive.

The following solvent diffusion method may be used when a fat soluble active agent is to be enclosed in ionic nanoparticles. PLGA (or PLA) may be dissolved in acetone along with the active agent. The acetone solution may be added dropwise to an aqueous solution that includes polyvinyl alcohol, Pluronic F68, or Tween 20 while being stirred. A fine particle emulsion can thus be obtained. The emulsion may be separated by using ultracentrifugation, and a resulting supernatant may be removed and washed with purified water. Alternatively, the following method (a solvent evaporation method) may also be adopted. PLGA (or PLA) may be dissolved in 1 mL of dichloromethane along with the active agent. An aqueous solution of added egg yolk lecithin and the dichloromethane solution may be mixed and stirred, and the resultant exposed to ultrasonic irradiation. The resultant may then be stirred at room temperature for 2 hours. Resultant fine particles may then be separated by using ultracentrifugation. A resultant supernatant may be removed and washed with purified water.

Non-limiting examples of active agents that may be enclosed in ionic nanoparticles include various cancer therapeutic agents, therapeutic genes, and peptides. Such active agents may be targeted to specific sites of a subject when enclosed in an ionic nanoparticle and delivered by iontophoresis. The active agents may also incorporate sustained release properties. Furthermore, the active agent may be administered by using iontophoresis even if the active agent itself does not readily dissociate into ions, and even if the active agent is substantially insoluble in water. When applied to an active agent having a large molecular weight, a valence greater than that of the original ionic charge can be provided for the entirety of the active agent by, for example, adjusting the number of functional groups on the nanoparticles, thus increasing mobility and facilitating increased transport.

Examples of cationic active agents that may be enclosed in ionic nanoparticles include: anesthetics (such as procaine hydrochloride and lidocaine hydrochloride); gastrointestinal disease therapeutics (such as carnitine chloride);

skeletal muscle relaxants (such as vancuronium bromide); and antibiotics (such as tetracycline based preparations, kanamycin based preparations, and gentamicin based preparations).

Examples of anionic active agents that may be enclosed in ionic nanoparticles include: vitamins (such as riboflavin phosphate, nicotinic acid, ascorbic acid, and folic acid); adrenal cortex hormones (such as a hydrocortisone based water soluble preparations, and dexamethasone based and prednisolone based water soluble preparations such as prednisolone sodium phosphate and dexamethasone sodium phosphate); and antibacterial agents (such as a quinolone based preparations).

Examples of vaccines that may be enclosed in ionic nanoparticles include BCG vaccine, hepatitis A vaccine, melanoma vaccine, measles vaccine, poliomyelitis vaccine, and influenza vaccines.

Examples of adjuvants that may be enclosed in ionic nanoparticles include MPL (Monophosphoryl lipid A), DMPC (dimyristoylphosphatidylcholine), QS-21, DDA (Dimethyl dioctadecyl ammonium chloride), and RC-529.

Furthermore, combinations of a vaccine and an adjuvant may also be enclosed, such as: a combination of a positively ionized vaccine and RC-529; a combination of a negatively ionized vaccine and DDA; a combination of a BCG vaccine and MPL; a combination of a hepatitis A vaccine and DMPC; and a combination of a melanoma vaccine and QS-21.

Other combinations of active agents may also be used, such as: a combination of a hypotensive active agent and a hypotensive diuretic agent, for example lisinopril and hydrochlorothiazide, methyldopa and hydrochlorothiazide, clonidine hydrochloride and chlorthalidone, and benazepril hydrochloride and hydrochlorothiazide; a combination containing an antidiabetic agent, such as insulin and metformin hydrochloride; and other combinations such as ozagrel hydrochloride and ozagrel sodium, and codeine hydrochloride and promethazine hydrochloride.

The amount of each ionic active agent present may be set to effect a desired blood concentration upon application to a subject over a certain period of time. The amount may be set by one skilled in the art in accordance with, for example, the size and/or thickness of an active agent solution reservoir, the size of an active agent release surface area, the size of an electric potential applied, administration time, or the like.

Conductive materials such as carbon and platinum may be used in the counter electrode of the electrode assembly.

The electrolyte solution reservoir may comprise a thin film capable of that holds with, and holding, an electrolyte solution. The thin film may comprise the same material as that used in an active agent solution reservoir, described below. It may be desirable to avoid use of electrolyte solutions that may tend to damage the biological interface of a subject, and instead to use an organic acid or salt thereof present in the metabolic cycle of a subject as the electrolyte solution. Typical examples include lactic acid and fumaric acid. Specifically, an aqueous solution of 1M of lactic acid and 1M of sodium fumarate (1:1) may be used. Such electrolyte solutions may have high solubility with respect to water, pass current well, and show small changes in pH at a constant current.

The active agent solution reservoir may comprise a thin film capable of that holds with, and holding, an ionic active agent or the like. Examples include hydrogel forms of acrylic resins, segmented polyurethane based gel matrix films, and ion conductive porous sheets for forming a gel matrix-like solid electrolyte (refer to a porous polymer disclosed in JP 11-273452 A using, as a base, an acrylonitrile copolymer containing 50 mol % or more, preferably 70 to 98 mol % or more of acrylonitrile and having a porosity of 20 to 80%). It may be advantageous to use an impregnation rate (defined as (W-D)/D, where D indicates dry weight and W indicates weight after impregnation) of 30 to 40%.

In addition, the active agent solution reservoir may have pores capable of holding ionic nanoparticles, and allowing ionic nanoparticles to pass therethrough. A porous structure may allow the reservoir to be immersed in a liquid containing the ionic nanoparticles, or allow the active agent solution reservoir to aspirate a liquid containing the ionic nanoparticles.

Examples of cation exchange membrane include NEOSEPTAs (CM-1, CM-2, CMX, CMS, CMB, and CLE04-2) manufactured by Tokuyama Co., Ltd. Examples of anion exchange membrane include NEOSEPTAs (AM-1, AM-3, AMX, AHA, ACH, ACS, ALE04-2, and AIP-21) manufactured by Tokuyama Co., Ltd. Cation exchange membranes may comprise a porous film having cavities, a portion or the entirety of which are filled with an cation exchange resin. Anion exchange membranes may comprise a porous film having cavities, a portion or the entirety of which are filled with an anion exchange resin.

Ion exchange membranes may have pores through which ionic nanoparticles can pass. In particular, an ion exchange membrane having the same polarity as that of the ionic nanoparticles used may have pores through which the ionic nanoparticles can pass, while an ion exchange membrane having a polarity opposite that of the ionic nanoparticles may comprise a non-porous membrane, allowing the ionic nanoparticles to be effectively supplied toward a biological interface.

The ion exchange resins may be fluorine based and include a perfluorocarbon skeleton having an ion exchange group, or may be hydrocarbon based and include a nonfluorinated resin as a skeleton. Hydrocarbon based ion exchange resins may be the easier of the two types to manufacture. The ion exchange resin may be filled into the porous film at from 5 to 95 mass %.

Ion exchange groups are not limited to functional groups having negative or positive charge when in aqueous solution. Functional groups may also be present in the form of a free acid or a salt. Examples of cation exchange groups include sulfonic groups, carboxylic acid groups, and phosphonic acid groups. Examples of counter cations for the cation exchange group include: alkali cations such as sodium ions and potassium ions, and ammonium ions. Examples of anion exchange groups include primary amino groups, secondary amino groups, tertiary amino groups, quaternary ammonium groups, pyridyl groups, imidazole groups, quaternary pyridium groups, and quaternary imidazolium groups. Examples of counter cations for the anion exchange group include: halogen ions such as chlorine ions, and hydroxy ions.

In addition, there are no specific limitations placed on film porosity. To satisfy both of high strength and flexibility, it may be advantageous that the porous film be made using a thermoplastic resin. Examples of the thermoplastic resins include: polyolefin resins such as homopolymers or copolymers of α-olefins, such as ethylene, propylene, 1-butene, 1-pentene, 1-hexene, 3-methyl-1-butene, 4-methyl-1pentene, and 5-methyl-1-heptene; vinyl chloride based resins such as polyvinyl chloride, vinyl chloride-vinyl acetate copolymers, vinyl chloride-vinylidene chloride copolymers, and vinyl chloride-olefin copolymers; fluorine based resins such as polytetrafluoroethylene, polychlorotrifluoroethylene, polyvinylidene fluoride, tetrafluoroethylene-hexafluoropropylene copolymers, tetrafluoroethylene-perfluoroalkyl vinylether copolymers, and tetrafluoroethylene ethylene copolymers; polyamide resins such as nylon 66; and polyimide resins. Polyolefin resins may have advantages in mechanical strength, flexibility, chemical stability, and chemical resistance.

Further, the mean pore size of the porous films may range from 0.005 to 5.0 μm. Mean pore size as used herein indicates mean flow pore size measured in conformance with the bubble point method (JIS-K3832-1990).

Similarly, the porosity of the porous film may be from 20 to 95%. In consideration of the thickness of an ion exchange membrane, the thickness of the porous film may be from 5 to 140 μm. An anion exchange membrane or a cation exchange membrane formed by using a porous film generally has the same thickness as that of the porous film, but may also have a thickness up to about 20 μm larger than that of the porous film.

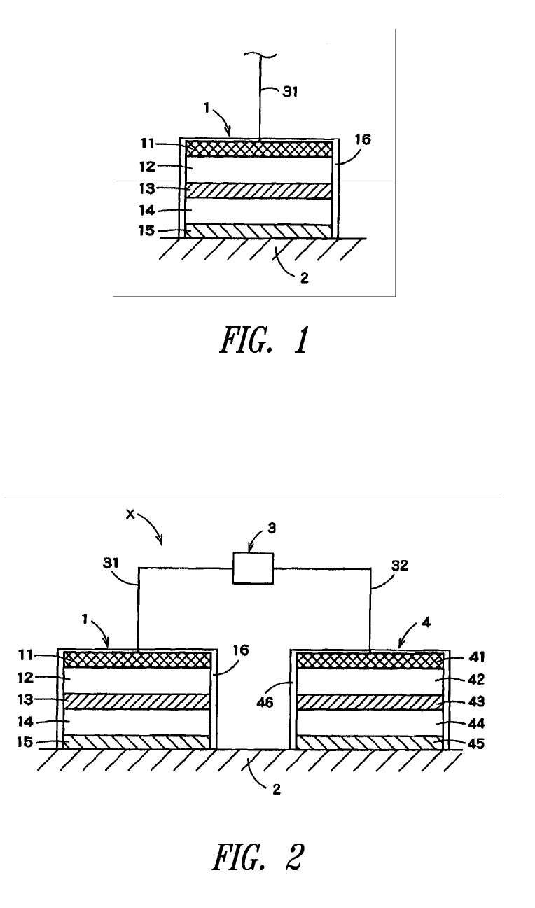

FIG. 1 is a schematic view showing an electrode assembly 1 for iontophoresis arranged on a biological interface 2. The electrode assembly 1 may be used as an active electrode assembly for transdermally administering an ionic active agent. The electrode assembly 1 may comprise: an electrode 11 coupled, through an electric cable or other conductive path 31, to an electric power source that has the same polarity as that of the charge of an ionic active agent; an electrolyte solution reservoir 12 that holds an electrolyte solution, the electrolyte solution reservoir 12 disposed adjacent to or proximate the electrode 11; an ion exchange membrane 13 that selectively passes ions having a polarity opposite that of the ionic active agent, the ion exchange membrane 13 adjacent to or proximate the electrolyte solution reservoir 12; an active agent solution reservoir 14 that holds the ionic active agent, the active agent solution reservoir 14 adjacent to or proximate the ion exchange membrane 13; and an ion exchange membrane 15 that selectively passes ions having the same polarity as that of the ionic active agent, the ion exchange membrane 15 adjacent to or proximate the active agent solution reservoir 14. A cover or container 16 may be used to house the electrode assembly 1.

FIG. 2 is a schematic view showing an iontophoresis device X comprising: the electrode assembly (active electrode assembly) 1; an electric power source 3; and a counter electrode assembly 4 disposed on the biological interface 2.

The electrode assembly 1 is coupled via the electric cable or other conductive path 31 to a pole of the electric power source 3 having the same polarity as that of the active agent. The counter electrode assembly 4 may comprise: an electrode 41 coupled via an electric cable or other conductive path 32 to a pole of the electric power source 3 having a polarity opposite to the charge of an ionic active agent; an electrolyte solution reservoir 42 that is that holds the electrolyte solution, the electrolyte solution reservoir 42 adjacent to or proximate the electrode 41; an ion exchange membrane 43 that selectively passes ions having the same polarity as that of the ionic active agent, the ion exchange membrane 43 adjacent to or proximate the electrolyte solution reservoir 42; an electrolyte solution reservoir 44 that holds the electrolyte solution, the electrolyte solution reservoir 44 adjacent to or proximate the ion exchange membrane 43; and an ion exchange membrane 45 that selectively passes ions having a polarity opposite that of the ionic active agent, the ion exchange membrane 45 adjacent to or proximate the electrolyte solution reservoir 44. The entirety of the counter electrode assembly 4 may be housed in a cover or container 46. Note that the counter electrode assembly 4 may also take on other configurations. For example, the electrolyte solution reservoir 42 and the ion exchange membrane may be omitted.

When an electrode assembly holding an ionic active agent is energized by the electric power source 3, the ionic active agent may move to a side opposite the electrode due to electrophoresis and/or electro-osmotic forces caused by an electric field, and thus be transdermally administered to a subject via the ion exchange membrane 15. The ion exchange membrane 13 disposed on the electrode side selectively passes ions having a polarity opposite that of the ionic active agent, thus substantially preventing movement of the ionic active agent toward the electrode.

A configuration that includes a plurality of active (or counter) electrode assemblies may also be employed. A plurality of different types of ionic active agents may thus be held by the active electrode assembly, and one or more of the ionic active agents may be enclosed in an ionic nanoparticle.

Alternatively, a plurality of electrode assemblies may be configured as active agent administering means and assembled integrally in one package for convenience of handling, for example. No particular limitations are placed on packaging materials in this case, provided that the packaging material does not substantially affect the administration of an ionic active agent. One example of a packaging material is polyolefin. Furthermore, current control means may be provided in order to administer a predetermined amount of an active agent within a predetermined time period. The active agent administering means, the current control means, and an electric power source device may be configured integrally. For example, a button battery may be used as the electric power source device, and an IC chip may be used as the current control means.

WO 03/037425 A1, the contents of which are hereby incorporated by reference in their entirety, by the applicant of the present disclosure describes specific elements in more detail.

The above description of illustrated embodiments, including what is described in the Abstract, is not intended to be exhaustive or to limit the embodiments to the precise forms disclosed. Although specific embodiments and examples are described herein for illustrative purposes, various equivalent modifications can be made without departing from the spirit and scope of the disclosure, as will be recognized by those skilled in the relevant art. The teachings provided herein of the various embodiments can be applied to other problem-solving systems devices, and methods, not necessarily the exemplary problem-solving systems devices, and methods generally described above.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Provisional Patent Application Ser. No. 60/730,226, filed Oct. 24, 2005, are incorporated herein by reference, in their entirety.

Aspects of the embodiments can be modified, if necessary, to employ systems, circuits, and concepts of the various patents, applications, and publications to provide yet further embodiments.

These and other changes can be made to the embodiments in light of the above-detailed description. In general, in the following claims, the terms used should not be construed to limit the invention to the specific embodiments disclosed in the specification and the claims, but should be construed to include all possible embodiments along with the full scope of equivalents to which such claims are entitled. Accordingly, the scope of the invention shall only be construed and defined by the scope of the appended claims.

What is claimed is:

1. An electrode assembly for iontophoresis, comprising;
an active agent enclosed in an ionic nanoparticle; and
an electrode operable to apply an electromotive force that drives the ionic nanoparticle from the electrode assembly.

2. The electrode assembly according to claim 1, further comprising:
an electric power source operable to apply an electrical potential to the electrode.

3. The electrode assembly according to claim 2, further comprising in order from an interior of the electrode assembly to an exterior thereof:
an electrolyte solution reservoir that holds an electrolyte solution, the electrolyte solution reservoir proximate the electrode;
a first ion exchange membrane that selectively passes ions having a polarity opposite that of the ionic nanoparticles;
an active agent solution reservoir that holds ionic nanoparticles; and
a second ion exchange membrane that selectively passes ions having the same polarity as that of the ionic nanoparticles.

4. The electrode assembly according to claim 3, wherein the active agent solution reservoir includes pores capable of holding and passing the ionic nanoparticle.

5. The electrode assembly according to claim 3, wherein the second ion exchange membrane includes pores capable of passing the ionic nanoparticle, and wherein the first ion exchange membrane does not include pores capable of passing the ionic nanoparticle.

6. The electrode assembly according to claim 1, wherein the ionic nanoparticle comprises a cationic nanoparticle.

7. The electrode assembly according to claim 1, wherein the ionic nanoparticle comprises an anionic nanoparticle.

8. The electrode assembly according to claim 1, wherein the active agent enclosed in the ionic nanoparticle is selected from the group consisting of cancer therapeutic agents, nucleic acids such as genes, and peptides.

9. An iontophoresis device comprising:
an electric power source device;
active agent administration means comprising two or more electrode assemblies, at least one of the electrode assemblies having a plurality of ionic nanoparticles and an electrode; and
current control means for controlling a current flow to the electrode assemblies from the electric power source device, current flow from the current control means releasing the ionic nanoparticle to be administered transdermally to a subject.

10. The iontophoresis device according to claim 9, wherein the active agent administration means is configured integrally.
